(19) United States
(12) Patent Application Publication
(10) Pub. No.: US 2008/0208098 A1
(43) Pub. Date: Aug. 28, 2008

(54) METHODS, APPARATUS, AND PRODUCTS FOR WOUND CARE

(76) Inventor: Lynda Rennix, Beaumont, TX (US)

Correspondence Address: J. M. (Mark) Gilbreth, P. O. Box 2428, Bellaire, TX, 77402-2428, US

(21) Appl. No.: 11/680,466

(22) Filed: Feb. 28, 2007

(51) Int. Cl. A61F 13/00

(52) U.S. Cl. 602/47

(57) ABSTRACT

A medical access cover having a first layer and a second layer. The first layer comprises medically sterile material and defining lines which define a plurality of flaps that fold open to define a first wound access opening. The second layer defines a second wound access opening. The first layer and second layer are positioned suitable to align the first opening and the second opening.

### BACKGROUND OF THE INVENTION

1. Field of the Invention

The present invention relates to wound care. In another aspect, the present invention relates to methods, apparatus, and products for wound care.

2. Description of the Related Art

Long-term access to a patient's bloodstream (longer than one month, for example) is required for many medical treatments including antibiotic therapy, hemodialysis access, chemotherapy regimens, and other treatments that require repeated infusion or blood processing.

Today, many people rely upon surgically implanted, indwelling catheters for their health and well being. For example, many diabetic patients use indwelling subclavian catheters for delivering insulin. Patients undergoing long term chemotherapy also may have an indwelling catheter. All peritoneal dialysis patients use an indwelling peritoneal catheter for delivering dialysis fluid.

Unfortunately a catheter wound, as with any wound to the skin, can increase the susceptibility of contamination or infection at the wound site, or sepsis of the patient due to bacteria or other micro-organisms entering the site or blood stream via the catheter.

Thus, protection of the catheter exit site is a significant daily task for these people. Infection of the catheter exit site must be avoided to prevent unintended removal and replacement of the catheter.

There are a number of patents and publications that relate to wound care, of which the following are merely a few.

U.S. Pat. No. 4,941,882, issued Jul. 17, 1990, to Ward et al., discloses a dressing for retaining a cannula on the skin. The dressing comprises a backing film coated on one face with an adhesive layer and with first and second release sheets covering the adhesive layer. A hole and a dividing line are cut through the dressing and second release sheet so that the dividing line extends from the hole to one edge of the dressing and the edge of the second release sheet. In use when the second release sheet is removed the hole in the dressing fits around the indwelling cannula. In a preferred form a portion of the dressing comprises an adhesive coated handle which is stiffer than the remainder of the dressing and which carries the hole and dividing line.

U.S. Pat. No. 5,495,856, issued Mar. 5, 1996, to Fentress, discloses a moisture barrier includes inner and outer moisture resistant layers. A moisture absorbing material occupies the region between the two layers. The peripheral edges of the inner and outer layers adhere to the intended surface surrounding the protected area. The adhered edge of the outer layer is spaced from the adhered edge of the inner layer, forming a gap between them. The gap communicates with the open region that contains the moisture absorbing material. The gap presents a path that diverts moisture away from adjoining portions of the seal and toward the moisture absorbing material. Preferably, the moisture absorbing material displays different visible characteristics, depending upon the presence or absence of moisture. The moisture sensing material is positioned for viewing through a light-transmissive portion of the outer barrier layer. The moisture barrier serves to affirmatively keep moisture away from a protected area. The moisture barrier can also serve to immediately notify the user when unwanted invasion of moisture threatens the protected area.

U.S. Pat. No. 5,605,546, issued Feb. 25, 1997, to Wolzinger et al, discloses an apparatus for protecting the outwardly extending portion, for example, the distal end, of an indwelling medical treatment device, for example, a catheter, are disclosed. Such apparatus include an at least partially transparent receptacle defining a chamber sized and adapted to receive an outwardly extending portion of an indwelling medical treatment device, an inlet in the receptacle through which the outwardly extending portion is passed to be received by the chamber, and a securement member on the receptacle adapted to be secured to the human or animal patient so as to substantially prevent liquid contamination of the outwardly extending portion received in the chamber.

U.S. Pat. No. 6,222,090, issued Apr. 24, 2001, to Weston, discloses a protective cover for keeping dry an area of an animal body during bathing, showering, or swimming is disclosed. One embodiment of the protective cover includes a flexible waterproof sheet having a waterproof adhesive provided around the edge of the sheet to affix the sheet to the body and prevent water or moisture from contacting the area to be kept dry. Another embodiment includes a cover having a raised area so the cover does not contact the area of the body to be kept dry. Methods of use are also described.

U.S. Pat. No. 6,276,3643, issued Aug. 21, 2001, to Warner, discloses a protective sleeve for a chronically-implanted intravenous (IV) site. The sleeve is made of a prophylactic waterproof material, and is normally worn by a person while showering to prevent moisture from invading the IV site. The sleeve in one embodiment has two elastomeric beads which form a watertight seal between a person's arm and the beads, and the sleeve billows in its mid-section so that it does not snag the catheter. The sleeve in another embodiment has a binding at one end and at least a binding at the other end integrally formed in the sleeve or in another embodiment a plurality of indentations axially spaced on a tapered portion of the sleeve.

U.S. Patent Application Publication No. 2002/0123710, published Sep. 5, 2002, to Worthley, discloses a film dressing for wounds and/or catheter sites and a method for applying such a dressing and a process for making the same are provided. Preferably, the film dressing has a substantially clear top layer. The top layer may be a moisture vapor semi-permeable film. The moisture vapor semi-permeable film may be coated with an adhesive and may cover a window cut out of a semi-permeable tape border having an adhesive layer and a hydrocolloid silver adhesive. The dressing may further have liners for use in the application of the dressing to a patient.

U.S. Patent Application Publication No. 2003/0216709, published Nov. 20, 2003, and U.S. Application Publication No. 2004/0267238, published Dec. 30, 2004, both to Haarala et al., disclose medical devices and related methods that enable physicians and/or other medical personnel to access percutaneously the interior of a patient. One particular device includes a housing that defines a cavity, a first opening into the cavity, and a second opening into the cavity. The housing is implantable in a patient. The cavity is placed subcutaneously within the patient. The first opening is substantially flush with the surface of the skin of the patient and creates a percutaneous passageway from the exterior of the skin of the patient into the cavity, and the second opening creates a passageway from the cavity into the interior of the patient. A connector is coupled to the second opening and disposed substantially within the cavity to enable a connection between a first device and a second device disposed within the interior of the patient.

In spite of the above patents and publications relating to wound care, there is still a need for improvement in wound care.

### SUMMARY OF THE INVENTION

The following presents a general summary of some of the many possible embodiments of the present invention in order to provide a basic understanding of the invention. This summary is not an extensive overview of all embodiments of the invention. This summary is not intended to identify key or critical elements of the invention or to delineate or otherwise limit the scope of the claims. The following summary merely presents some concepts of the invention in a general form as a prelude to the more detailed description that follows.

According to one embodiment of the present invention, there is provided a medical access cover. The cover has a first layer comprising medically sterile material and defining lines which define a plurality of flaps that fold open to define a first wound access opening. The cover has a second layer defining a second wound access opening. The first layer and second layer are positioned suitable to align the first opening and the second opening.

According to another embodiment of the present invention, there is provided a method of treating a wound. The method includes placing a medical access cover over the wound, wherein the medical access cover comprises a first layer comprising medically sterile material and defining lines which define a plurality of flaps that fold open to define a first wound access opening. The method may also include folding open the flaps to define a first wound access opening.

### BRIEF DESCRIPTION OF THE DRAWINGS

The following drawings illustrate some of the many possible embodiments of this disclosure in order to provide a basic understanding of this disclosure. These drawings do not provide an extensive overview of all embodiments of this disclosure. These drawings are not intended to identify key or critical elements of the disclosure or to delineate or otherwise limit the scope of the claims. The following drawings merely present some concepts of the disclosure in a general form. Thus, for a detailed understanding of this disclosure, reference should be made to the following detailed description, taken in conjunction with the accompanying drawings, in which like elements have been given like numerals.

FIG. 1 is a non-limiting illustration of one embodiment of medical access cover 100, comprising layer 101 having 4 perforation lines 103 extending radially away from point 102.

FIG. 2 is a non-limiting illustration of medical access cover 100 of FIG. 1 in which flaps 105 have been folded and secured onto layer 101.

FIG. 3 is a non-limiting illustration of another embodiment of medical access cover 100, and includes 3 perforations 103 radially extending from point 102, which define 3 flaps 105.

FIG. 4 is a non-limiting illustration of medical access cover 100 of FIG. 3 in which flaps 105 have been folded and secured onto layer 101.

FIG. 5A is a non-limiting illustration showing a one layer embodiment of medical access cover 100 having base flap layer 101.

FIG. 5B is a non-limiting illustration showing an embodiment of medical access cover 100 having a base flap layer 101, and at least one top layer 201.

FIG. 5C is a non-limiting illustration showing an embodiment of medical access cover 100 having a base flap layer 101, and at least one bottom layer 401.

FIG. 5D is a non-limiting illustration showing an embodiment of medical access cover 100 having a base flap layer 101, at least one top layer 201, and at least one bottom layer 401.

FIG. 6 is a non-limiting illustration showing that top layer 201 may be designed with flaps as shown for base flap layer 101, or it may merely have a complimentary opening 211.

FIG. 7 is a non-limiting illustration showing that bottom layer 401 may be designed with flaps as shown for base flap layer 101, or it may merely have a complimentary opening 411.

### DETAILED DESCRIPTION SOME EMBODIMENTS OF THE INVENTION

The following presents a detailed description of some of the many possible embodiments of the present invention in order to provide a basic understanding of the invention. The following is not an extensive overview of all embodiments of the invention.

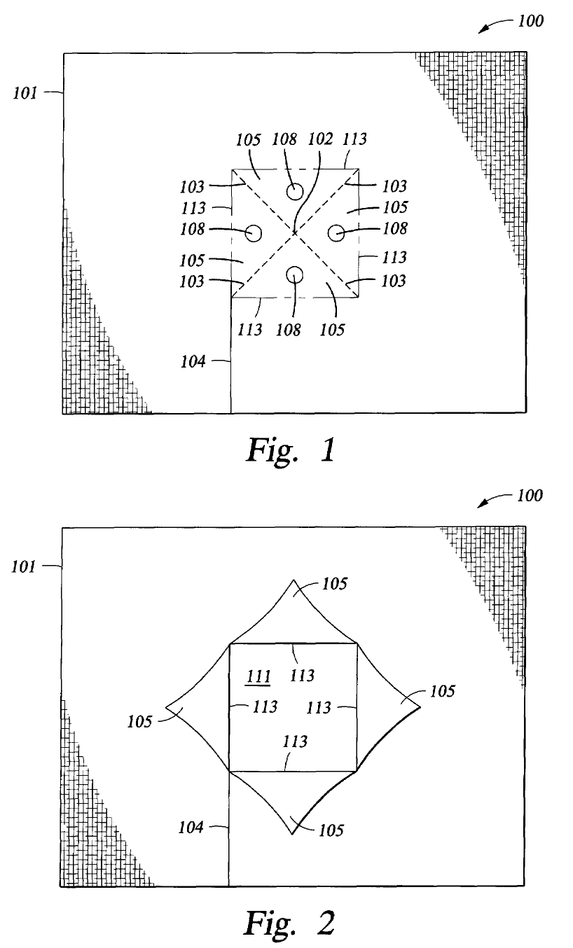

Referring now to FIG. 1, there is provided a non-limiting illustration of one embodiment of medical access cover 100, comprising top layer 101 having a plurality of lines 103 extending radially away from point 102. These 4 lines define 4 flaps 105 which may be folded onto layer 101. Lines 103 may be score lines, guide lines for cutting or tearing, perforation lines, or pre-cut lines. The tearing/cutting of layer 101 along the guide, score, or perforation lines 103 creates flaps 105, which may then be folded onto layer 101. Optional adhesive members 108 on flaps 105 may be used to secure flaps 105 to a complimentary mating member on layer 101. One or more dividing lines 104 may be provided to allow placement of medical access cover 100 around catheter and other type lines that might be attached to a patient.

Referring additionally to FIG. 2, there is shown medical access cover 100 of FIG. 1 in which flaps 105 have been folded and secured onto layer 101. The boundaries of access opening 111 are defined by fold lines 113. These fold lines 113 are formed when flaps 105 are folded onto layer 101.

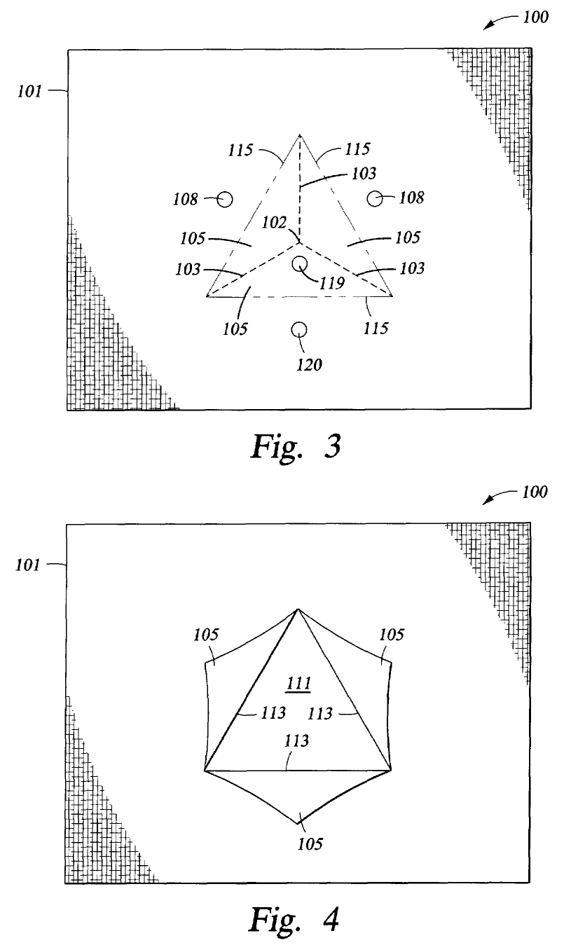

Referring now to FIG. 3, there is shown an illustration of another embodiment of medical access cover 100. Layer 101 includes 3 perforations radially extending from point 102, which define 3 flaps 105. As in the above embodiment, tearing layer 101 at the perforation lines creates flaps 105, which may then be folded onto layer 101. Layer 101 is also provided with stitching 115 to predispose the formation of fold line 113 at stitching 115.

As illustrated, adhesive members 108 may be positioned on layer 101 rather than on flap 105. In such an instance flaps 105 are folded onto adhesive member 108 and thus secured. As further illustrated, a pair of mating connectors 119 on flap 105 and 120 on layer 101 may be provided to secure flap 105 to layer 101. Any suitable type of pairs of mating connectors may be utilized, from interlocking snaps, magnetic pairs, adhesive pairs, to hook-and-loop pairs (a commercial example of which is sold under the brand name VELCRO).

Referring additionally to FIG. 4, there is shown medical access cover 100 of FIG. 3 in which flaps 105 have been folded and secured onto layer 101. Access opening 111 is defined by fold lines 113 formed when flaps 105 are folded onto layer 101.

It should be clear that any number of flaps 105 may be defined by any number of perforations 103 in layer 101. It should also be understood that any shape and size of flap 105 may be provided by providing the appropriate corresponding perforations 103 for the shape and size of flap 105 desired. And, for any given medical access cover, it is not required that all of the flaps be of the same size and shape.

In the practice of the present invention, it should be understood that base flap layer 101 may serve one or more functions. Non-limiting examples of such functions include moisture barrier, air barrier, insulation layer, aeration layer, absorption layer, disinfecting layer (a disinfectant incorporated therein), pharmaceutical release layer (a pharmaceutical incorporated therein for absorption into the skin, odor absorption layer, odor release layer (pleasant odorant incorporated therein), padding layer (to pad/protect against incidental contract), cooling layer (cooling material incorporated therein or circulated there thru), heating layer (heating material incorporated therein or circulated there thru), vibrating layer (vibrating element incorporated therein), and moisture layer (a liquid or gel incorporated therein for release).

Any suitable materials may be selected for base flap layer 101. There are a number of cloths and plastic materials which have found acceptance in the medical arts for us in medical bandage applications, and those will be suitable here too.

The backside of medical device cover 100 which will contact a patient's skin, may be sufficiently treated to promote comfortable contact with skin, including having padding, smooth texture, and the like. The backside of medical device cover 100 may also be adhesive to allow for secure contact with a patient's skin.

Base flap layer 101 may be of any suitable dimensional shape as the particular medical application requires. Base flap layer 101 may be supplemented with one or more backing layers, and one or more covering layers, as desired.

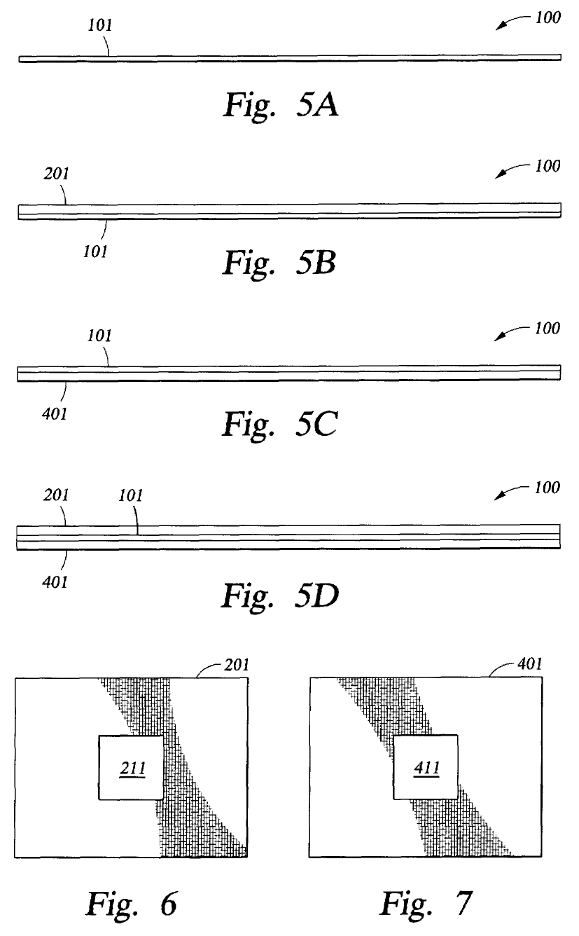

Referring now to FIGS. 5A, 5B, 5C, and 5D, there are shown non-limiting side views of a number of non-limiting embodiments of medical access cover 100.

FIG. 5A shows a one layer embodiment of medical access cover 100 having base flap layer 101. In this embodiment, medical access cover comprises only base flap layer 101 as described herein.

FIG. 5B shows an embodiment of medical access cover 100 having a base flap layer 101, and at least one top layer 201. It should be understood that if medical access cover includes top layers 201, that any suitable number of top layers 201 may be utilized. The number of top layers 201 may be dictated by the desired use and function of top layers 201, and by any weight and space requirements. Thus, should top layers 201 be desired, it might be possible to have from 1 to 10 or more top layers 201, although in most practical instances there may be from 1 to 5 top layers 201, and in even more practical instances there may be from 1 to 3 top layers 201.

This top layer 201 may be designed with flaps as shown for base flap layer 101, or it may merely have a complimentary opening 211 as shown in FIG. 6. Thus, for a medical access cover 100 having a top layer 201, access opening 111 and access opening 211 will align and allow access to a wound. Top layer 201, as with all other layers which may be utilized with medical access cover 100, may have one or more dividing lines, such as dividing line 104 of layer 101.

FIG. 5C shows an embodiment of medical access cover 100 having a base flap layer 101, and at least one bottom layer 401. It should be understood that if medical access cover includes bottom layers 401, that any suitable number of top layers 401 may be utilized. The number of top layers 401 may be dictated by the desired use and function of top layers 401, and by any weight and space requirements. Thus, should top layers 401 be desired, it might be possible to have from 1 to 10 or more top layers 401, although in most practical instances there may be from 1 to 5 top layers 401, and in even more practical instances there may be from 1 to 3 top layers 401.

As with top layer 201, this bottom layer 401 may be designed with flaps as shown for base flap layer 101, or it may merely have a complimentary opening 411 as shown in FIG. 7. Thus, for a medical access cover 100 having a bottom layer 401, access opening 111 and access opening 411 will align and allow access to a wound.

In other non-limiting embodiments, base flap layer 101 access opening 113 may be a flap-free complimentary opening as shown in FIGS. 6 and 7 for the top and bottom layers. There may be instances where it is desired that base layer 101 have an opening free of any flaps. In such an instance this flap-free opening 113, as well as complimentary openings 211 and 411, may have a finish around the perimeter of their respective openings.

FIG. 5D shows an embodiment of medical access cover 100 having a base flap layer 101, at least one top layer 201, and at least one bottom layer 401. It should be understood that for a medical access cover 100 having both a top layer 201 and a bottom layer 401, access openings 111, 211 and 411 will align and allow access to a wound.

As with base flap layer 101, it should be understood, that any of the top layers 201 and the bottom layers 401 may serve one or more functions. As with base flap layer 101, non-limiting examples of such functions include moisture barrier, air barrier, insulation layer, aeration layer, absorption layer, disinfecting layer (a disinfectant incorporated therein), pharmaceutical release layer (a pharmaceutical incorporated therein for absorption into the skin, odor absorption layer, odor release layer (pleasant odorant incorporated therein), padding layer (to pad/protect against incidental contract), cooling layer (cooling material incorporated therein or circulated there thru), heating layer (heating material incorporated therein or circulated there thru), vibrating layer (vibrating element incorporated therein), and moisture layer (a liquid or gel incorporated therein for release).

It is envisioned that various top layers 201 and/or bottom layers 401 may be selected to compliment a base flap layer 101 and build a medical access cover as desired. For example, a kit may contain base flap layers and a number of other layers of various functions which are then mix-and-matched with the base flap layers as desired. In such a mix-and-match scenario, the various layers may just be stacked together to form a loose arranged medical access cover 100, or for a more secure arrangement and adhesive may be utilized, the layers may be clipped or stapled together, the layers may be equipped with adhesive materials on one or both sides, or mechanical connectors may be utilized.

In other non-limiting embodiments, a multi-layer medical access cover 100 may also be laminated together, sewn together, adhered together, or otherwise permanently formed together into a unitary item.

The present disclosure is to be taken as illustrative rather than as limiting the scope or nature of the claims below. Numerous modifications and variations will become apparent to those skilled in the art after studying the disclosure, including use of equivalent functional and/or structural substitutes for elements described herein, use of equivalent functional couplings for couplings described herein, and/or use of equivalent functional actions for actions described herein. Any insubstantial variations are to be considered within the scope of the claims below.

What is claimed is:

1. A medical access cover comprising:
A first layer comprising medically sterile material and defining lines that define a plurality of flaps that fold open to define a first wound access opening;
A second layer defining a second wound access opening;
Wherein the first layer and second layer are positioned suitable to align the first opening and the second opening.

2. The medical access cover of claim 1, further comprising:
A first member of a connector pair positioned on at least one of the flaps; and
A second member of the connector pair positioned on the first layer.

3. The medical access cover of claim 1, wherein the first layer comprises at least one of a moisture barrier, air barrier, insulation layer, aeration layer, absorption layer, disinfecting layer, pharmaceutical release layer, odor absorption layer, odor release layer, padding layer, cooling layer, heating layer, vibrating layer, and moisture layer.

4. The medical access cover of claim 3, further comprising:
A third layer defining a third wound access opening;
Wherein the third layer is positioned on the first layer, and wherein the first layer, second layer and third layer are positioned suitable to align the first opening, the second opening and the third opening.

5. The medical access cover of claim 4, wherein at least one of the first layer, the second layer or the third layer, comprises at least one of a moisture barrier, air barrier, insulation layer, aeration layer, absorption layer, disinfecting layer, pharmaceutical release layer, odor absorption layer, odor release layer, padding layer, cooling layer, heating layer, vibrating layer, and moisture layer.

6. A method of treating a wound, the method comprising:
placing a medical access cover over the wound, wherein the medical access cover comprises a first layer comprising medically sterile material and defining lines which define a plurality of flaps that fold open to define a first wound access opening; and
folding open the flaps to define a first wound access opening;

7. The method of claim 6, wherein the cover further comprises:
A first member of a connector pair positioned on at least one of the flaps;
A second member of the connector pair positioned on the first layer.

8. The method of claim 6, wherein the first layer comprises at least one of a moisture barrier, air barrier, insulation layer, aeration layer, absorption layer, disinfecting layer, pharmaceutical release layer, odor absorption layer, odor release layer, padding layer, cooling layer, heating layer, vibrating layer, and moisture layer.

9. The method of claim 8, wherein the cover further comprises:
A third layer defining a third wound access opening;
Wherein the third layer is positioned on the first layer, and wherein the first layer, second layer and third layer are positioned suitable to align the first opening, the second opening and the third opening.

10. The method of claim 9, wherein at least one of the first layer, the second layer or the third layer, comprises at least one of a moisture barrier, air barrier, insulation layer, aeration layer, absorption layer, disinfecting layer, pharmaceutical release layer, odor absorption layer, odor release layer, padding layer, cooling layer, heating layer, vibrating layer, and moisture layer.

11. A wound treatment kit comprising:
A first layer comprising medically sterile material and defining lines which define a plurality of flaps that fold open to define a first wound access opening; and,
A second layer defining a second wound access opening, wherein wherein the first layer comprises at least one of a moisture barrier, air barrier, insulation layer, aeration layer, absorption layer, disinfecting layer, pharmaceutical release layer, odor absorption layer, odor release layer, padding layer, cooling layer, heating layer, vibrating layer, and moisture layer.
